(19) United States
(12) Patent Application Publication
(10) Pub. No.: US 2014/0288595 A1
(43) Pub. Date: Sep. 25, 2014

(54) SOFT TISSUE INTERFERENCE FIT ANCHOR SYSTEM

(71) Applicant: Rajiv D. Pandya, Atlanta, GA (US)

(72) Inventor: Rajiv D. Pandya, Atlanta, GA (US)

(21) Appl. No.: 14/220,902

(22) Filed: Mar. 20, 2014

Related U.S. Application Data
(60) Provisional application No. 61/803,646, filed on Mar. 20, 2013.

(51) Int. Cl. A61B 17/04

(52) U.S. Cl. CPC A61B 17/0401; A61B 2017/0464; A61B 2017/0445; A61B 2017/0417; A61B 2017/045
USPC 606/232

(57) ABSTRACT

The soft tissue anchor system preferably has a sleeve (22) with a snap lock (21) and an insertable anchor stop (50) that permits the sutures (40) to be held under tension on insertion of the stop anchor (50).

### FIELD OF THE INVENTION

The present invention relates to devices used in arthoscopic surgical procedures and, more specifically, to an anchor system for securing soft tissue onto a bone.

### BACKGROUND OF THE INVENTION

There are a myriad of devices available for securing soft tissue such as a ligament to a bone. Some known devices include a sleeve which is inserted into a tunnel in the bone. The sleeve provides footing in the bone and is retained in the tunnel with threads or spikes. A tack or stud, configured for attaching to the soft tissue, may then be threaded into the sleeve.

Another example of a system for attaching a soft tissue to a bone is U.S. Pat. No. 5,964,764 to West et al. In West, a sheath passes through a tunnel in the bone. A portion on one end of the sheath overlaps and engages the surface of the bone. Soft tissue is then attached to the other end of the sheath. A pin with a cam member is slidably disposed relative to the sheath to urge the sheath to engage the bone. A system of sutures is used to pull the sheath through the tunnel and to withdraw the pin into the sheath. The system of sutures is severed and removed after inserting the anchoring system into the tunnel.

However, these known anchoring systems are difficult to use with an arthroscopic camera. In particular, it is difficult to thread sutures and tie knots during arthoscopic surgery without additional open incisions. Typically, when inserting these known anchoring systems, it is easier to perform open surgery to permit the threading of sutures and the tying of knots.

Furthermore, the current state of the art does not provide a way to maintain tension on the sutures secured to the soft tissue as an anchor is being fixed to the bone or thereafter. This is particularly true when a threading or tying of knots is required to hold the suture in place.

Therefore, there is a need in the art for a simpler anchoring system for use with arthoscopic surgical techniques. This new anchoring system must attach bodily tissue to a bone without additional open incisions and most preferably provide an anchoring of sutures in the absence of knots or tying while the sutures are held in tension.

### SUMMARY OF THE INVENTION

The present invention solves the above-identified problem by providing an improved soft tissue anchoring system. The improved soft tissue anchoring system provides a method of attaching bodily tissue to a bone during arthoscopic surgery without the need for additional open incisions.

Generally described, the soft tissue anchoring system of the present invention includes at least one suture secured to the soft tissue. The at least one suture is pulled through one end of a tunnel in the bone. A stop anchor is received through the other end of the tunnel to urge the suture toward the side of the tunnel. The stop anchor may take a variety of forms such as a cork, grommet or plug shape, and those skilled in the art will appreciate that the present invention is not limited to a particular shape of stop anchor, but may be enhanced based on the shape of the stop anchor and the particular application of the invention. The suture becomes wedged between the stop anchor and the tunnel, thereby resistively anchoring the soft tissue to the bone by providing an interference fit.

In one aspect of the present invention, the anchoring system further includes an elongated sleeve sized to be received and maintained within the tunnel. In an exemplary embodiment, the interior of the sleeve is tapered. Once the suture is received through the end of the sleeve, the stop anchor is then received through the other end of the sleeve to urge the suture toward the side of the sleeve so that the suture becomes wedged between the stop anchor and the sleeve, thereby anchoring the soft tissue to the bone.

In a more preferred embodiment, the suture anchoring assembly allows a tension to be applied to the sutures to eliminate looseness or slack and further provides a positive indication of proper suture anchoring to the surgeon.

A suture anchor system for attachment of a soft tissue to a bone tunnel with one or more sutures, the suture anchor has an elongated sleeve. The sleeve has a shaft with a central opening extending the length of the sleeve and a snap lock extending radially inwardly from the central opening, a stop anchor sized to be press fitted into the shaft central opening past the snap lock and wherein the one or more sutures secured to the soft tissue are held under tension as the stop anchor is press fit into the sleeve past the snap lock thereby fixing the one or more sutures along a length between the sleeve and the stop anchor.

The one or more sutures are further held at the stop anchor and the snap lock forming a bend in the suture. The snap lock can be located at an end of the sleeve and the stop anchor has an end positioned to be abutted against the snap lock. Alternatively, one or more snap locks can be located internal between opposing ends of the sleeve. The stop anchor, when pressed into the sleeve at each snap, locks the suture while forming a bend. The sleeve may have one or a plurality of internal snap locks and the anchor has one or a plurality of complimentary locking ridges or recesses positioned to lock at a predetermined insertion depth of the anchor. The elongated sleeve has a first end and a second end, the first end having a stop plate extending radially outwardly from the sleeve, the stop plate limits the insertion depth of the sleeve into the bone tunnel and lies on the exterior at the surface of the bone. The shaft can have a smooth exterior surface. Alternatively, the shaft can have a plurality of bone holding protrusions for fixing the sleeve in the bone tunnel. The plurality of protrusions can be spaced rings allowing the sleeve to be pressed into place inside the bone tunnel. The sleeve preferably has a stop plate to limit depth of insertion. The stop anchor has a rounded, tapered or conical entry end or tip. The stop anchor has a body extending from the entry end or tip to a distal end, the exterior surface between the ends sized to have an interference fit with internal surfaces of the shaft of the elongated sleeve over at least a length of 25 percent of the length of the anchor. The stop anchor and the shaft have a contacting interference fit along the length of the body from a first contact located at or near the entry end or tip to the distal end. The sutures on assembly are held and the stop anchor is pressed into the sleeve past the snap lock creating an audible or physical snap signaling a completed insertion. The stop anchor has a recessed hole at a distal end, the recessed hole sized to accommodate an insertion tool. The recessed hole can be a hexagonal, square, star, splined or round shape sized to receive a complimentary insertion tool.

A method for anchoring soft tissue to a bone, the bone having a tunnel therethrough is achieved by use of the anchor system of the present invention. The method has the steps of: inserting an elongated sleeve in the tunnel to be maintained between said stop anchor and the tunnel; attaching one or more sutures to the soft tissue; receiving said sutures in the tunnel; receiving a stop anchor in the tunnel by sliding disposing said stop anchor in said elongated sleeve in the tunnel, tensioning or tightening said sutures to remove slack and urging said sutures toward side walls of the tunnel with said stop anchor; and in response to said urging step, wedging said one or more sutures between said stop anchor and the elongated sleeve in the tunnel, thereby anchoring the soft tissue to the bone. The method may further have, in response to wedging said suture, the step of maintaining said suture between said stop anchor and said elongated sleeve. The wedging step may have said stop anchor providing substantially uniform contact with said elongated sleeve. The step of sliding disposing said stop anchor in said elongates sleeve further may have pressing said stop anchor past one or more snap locks to create a bend in said one or more sutures. The suture anchor system for attachment of a soft tissue to a bone tunnel with one or more sutures wherein the stop anchor is held at a distal end by an insertion tool and the surgeon completes assembly when he feels or hears the stop anchor pass the snap lock indicating a locked assembly.

The foregoing has broadly outlined some of the more pertinent aspects and features of the present invention. These should be construed to be merely illustrative of some of the more prominent features and applications of the invention. Other beneficial results can be obtained by applying the disclosed information in a different manner or by modifying the disclosed embodiments. Accordingly, other aspects and a more comprehensive understanding of the invention may be obtained by referring to the detailed description of the exemplary embodiments taken in conjunction with the accompanying drawings, in addition to the scope of the invention defined by the claims.

### BRIEF DESCRIPTION OF THE DRAWINGS

The invention will be described by way of example and with reference to the accompanying drawings in which:

FIG. 1 illustrates a cross-sectional view of one embodiment of an anchoring system of the present invention for securing soft tissue to a bone.

FIG. 2 illustrates a perspective view of one embodiment of an elongated sleeve useable in an exemplary embodiment of the present invention.

FIG. 3 illustrates a cross sectional view of the elongated sleeve of FIG. 2 inserted into a tunnel through the bone.

FIG. 4 illustrates one embodiment of a stop anchor of the present invention wedged into the elongated sleeve.

FIG. 5 illustrates an alternative embodiment of the anchoring system of the present invention wherein the stop anchor is wedged in the tunnel through the bone without the elongated sleeve.

FIG. 6 is a cross sectional view of the stop anchor shown abutted against a snap lock showing a bend in the suture according to one embodiment of the present invention.

FIG. 7 is a cross sectional view of an alternative elongated sleeve of the present invention.

FIG. 8 is a cross sectional view of another embodiment.

FIGS. 9A-9E show a top view of a recessed hole for holding the stop anchor on assembly.

FIG. 10 is a hand held exemplary device for inserting the stop anchor.

### DETAILED DESCRIPTION OF THE INVENTION

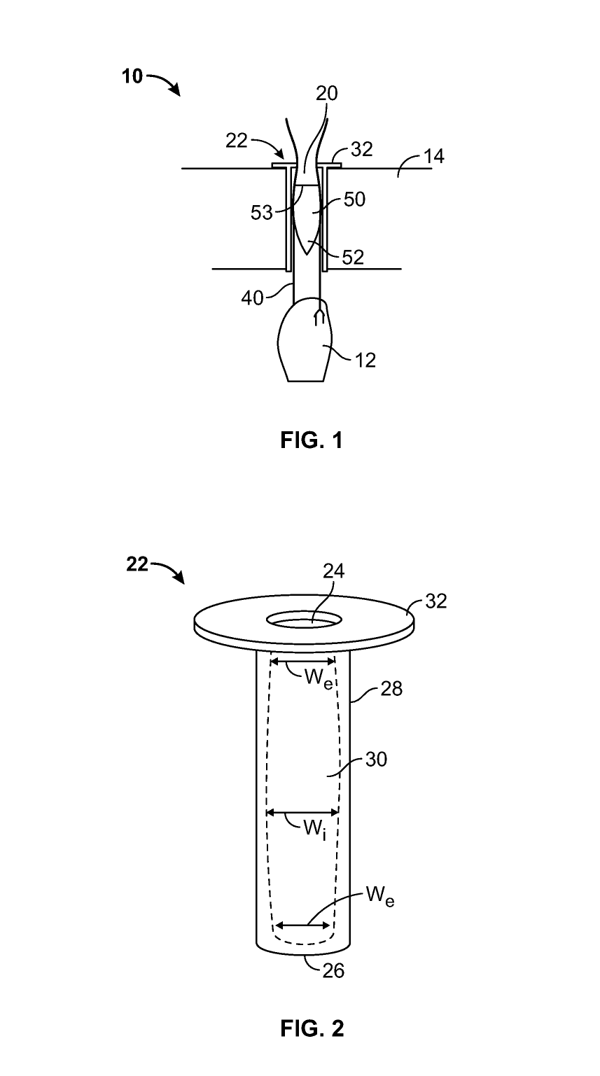

Referring now to the drawings in which like numerals indicate lit throughout the several views, FIG. 1 depicts one embodiment of an anchoring system 10 of the present invention. The anchoring system 10 is used during arthoscopic surgery for securing soft tissue 12 onto a bone 14 without requiring additional open incisions. Soft tissue includes, but is not limited to, ligaments, tendons or like grafts capable of being secured to a bone. Soft tissue also includes prosthetic soft tissue such as prosthetic ligaments.

In FIG. 1, the anchoring system 10 secures soft tissue 12 to a bone 14. The bone 14 includes a pre-formed tunnel 20 therethrough. The tunnel 20 is formed in a manner well known in the art. Because the quality of the bone 14 defining the tunnel 20 is variable, the anchoring system 10 preferably may include an elongated sleeve 22 made of a somewhat resilient surgically acceptable material and configured to be received and maintained within the tunnel 20. The outer diameter of the sleeve 22 corresponds with the diameter of the tunnel 20 so that the sleeve 22 presses tightly against the bone 14.

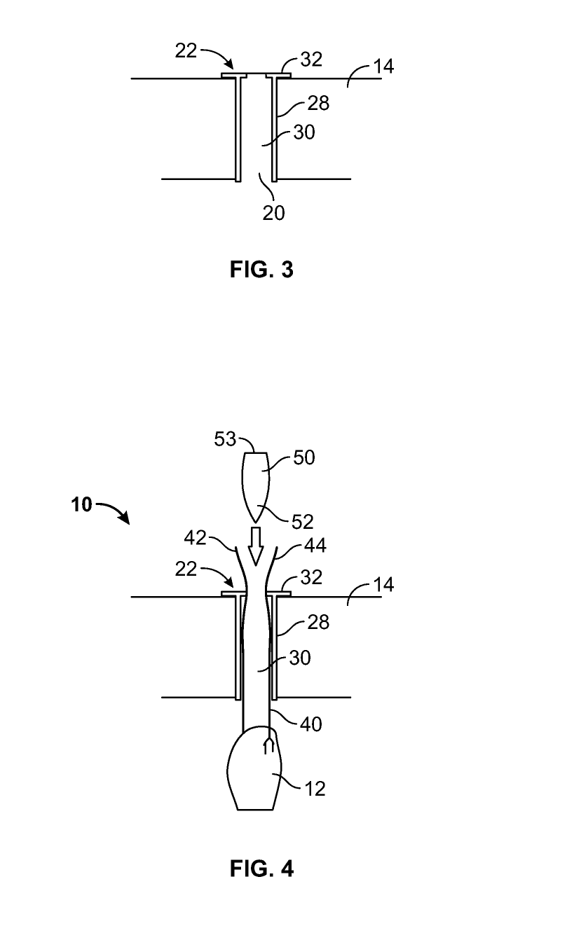

As shown in FIG. 2, the elongated sleeve 22 includes first and second ends 24 and 26, and a bore or shaft 28. The bore or shaft 28 includes a tapered inner channel portion 30 between ends 24 and 26. Preferably, the first and second ends 24, 26 have a reduced bore width wₑ compared to a bore width wᵢ of the inner channel portion 30. The elongated sleeve 22 further includes a lip 32 extending laterally from first end 24. As best shown in FIG. 3, the lip 32 overlaps the surface of the bone 14 when the elongated sleeve 22 is inserted in the tunnel 20.

The anchoring system 10 further includes one or more sutures, preferably at least one non-bioabsorbable suture 40 secured to the soft tissue 12. The manner in which the suture 40 is attached to the soft tissue 12 is commonly referred to as being looped through the soft tissue 12. Ends 42 and 44 of the suture 40 are received in the sleeve 22 through the second end 26, and pulled all the way through the sleeve 22 and the first end 24 as best shown in FIG. 4. It is critical to note that the suture 41 not require a knot in any manner.

The anchor system 10 further includes a stop anchor 50 which is also made o somewhat resilient surgically acceptable material and configured to be received and maintained within the sleeve 22. The stop anchor 50 may take a variety of forms such as a cork, grommet or plug shape, and those skilled in the art will appreciate that the present invention is not limited to a particular shape of stop anchor, but may be enhanced based on the shape of the stop anchor and the particular application of the invention. The stop anchor 50 is slidingly disposed in the sleeve 22 and urges the s 40 against the sleeve 22 to maintain the suture 40 in the sleeve 22. FIG. 1 illustrates the suture 40 wedged in the sleeve 22 between the stop anchor 50 and the sleeve 22. Note that the suture 40 is kept to the side of the sleeve 22 with the majority of the bore or shaft 28 occupied by the stop anchor 50. In one embodiment, the stop anchor is configured to be maintained within the sleeve 22 without a portion of the 50 extending beyond the sleeve 22 and, as best shown in FIGS. 1 and 4, the soft tissue 12 is free of the tunnel 20 and sleeve 22.

Preferably, the stop anchor 50 includes an ogive portion 52 so that force is applied in substantially a uniform radial manner to the inside of the sleeve 22. In one embodiment, the sleeve 22 is made of a resilient material, and thus the sleeve 22 outwardly expands when urged by the stop anchor 50. Therefore, the sleeve 22 is more tightly fitted within the tunnel 20. Moreover, because of the resilient properties the sleeve 22, the sleeve 22 is prevented from permanently deforming. In another embodiment, the sleeve 22 is rigid, thereby providing a solid hold within softer bone structures.

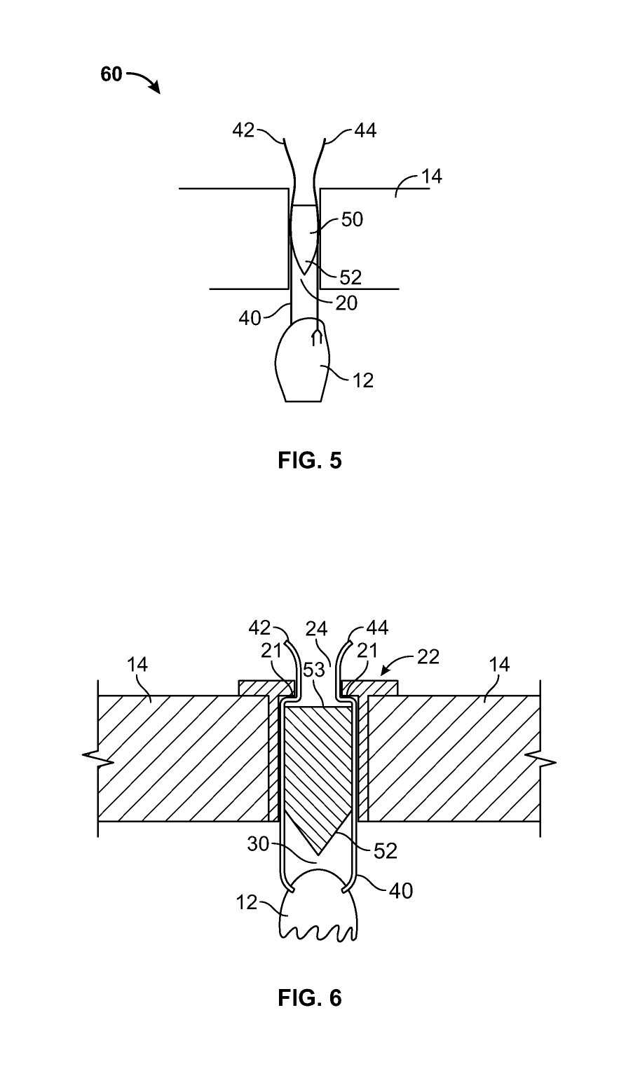

FIG. 5 illustrates an alternative embodiment of an anchoring system 60. The anchoring system 60 is distinguishable from the anchoring system 10 in that the anchoring system 60 does not utilize the sleeve 22 and instead relies upon the tunnel 20 for wedging the suture 40 with the stop anchor 50. The anchoring system 60 may be utilized instead of the anchoring system 10 when the quality of bone in the tunnel is high. The stop anchor 50 is configured to be received and maintained within the tunnel 20 with the suture 40 between the stop anchor 50 and the tunnel 20. The stop anchor 50 does not extend beyond the tunnel 20 and the soft tissue 12 is kept free of the tunnel 20. Also, the tunnel 20 may be tapered in a manner similarly described above in relation to sleeve 22.

In an alternative embodiment, multiple tunnels through the bone may be utilized to secure the sutures in place. This embodiment of the present invention advantageously allows the invention to be utilized even with smaller bones or in areas where soft tissue may be stretched or pulled in a variety of manners. In this embodiment, a single thread of the suture may be interference fit within the tunnel, or multiple suture loops may be used to secure the soft tissue to the bone.

One very important aspect of the present invention as shown in FIG. 1a is the sleeve can preferably have a snap lock 21 as shown in FIGS. 1 and 6. The snap lock 21, as shown, is an annular ring 21 protruding inwardly from the opening 24. As the stop anchor 50 is inserted into the sleeve 22, the body of the stop anchor 50 extending to the second or distal end 53 opposite the tip end at the ogive portion 52 or insertion end must be pressed through the snap lock 21 as the distal end 53 of the stop anchor 50 clears the snap lock 21 the surgeon will either hear an audible click sound or will feel the snap signaling the stop anchor 50 is properly seated in the elongated sleeve 22. As shown in FIG. 6, when this occurs the one or more sutures 40 are not only wedged along the sides of the stop anchor 50 and the sleeve 22, but are also bent between the snap lock 21 and the distal end 53. This creates a secondary fixation of the sutures 40. This signaling of proper fit is very helpful in that the surgeon can be assured the stop anchor 50 is fully seated. In FIG. 1, the stop anchor 50 is shown inserted well past the snap lock 21. While this is acceptable, the surgeon must limit his insertion depth to avoid going too deep. In the preferred embodiment, a snap lock 21 is used to signal the proper depth of insertion.

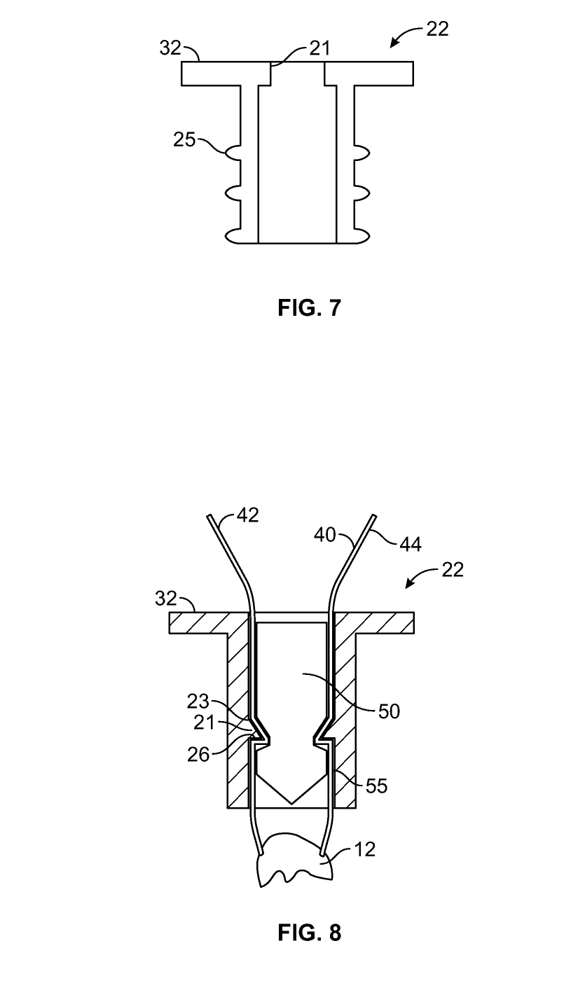

With reference to FIG. 7, an alternative elongated sleeve 22 is shown. In this embodiment, the sleeve 22 has a plurality of annular ridges 25 on the exterior surface to help hold the sleeve 22 in soft bone. In this embodiment, the sleeve 22 is held in place by the ridges.

In FIG. 8, another alternative embodiment is shown wherein the sleeve 22 has a snap lock 21 feature positioned between the ends of the sleeve 22. The snap lock 21 feature being an annular protrusion with a ramped first surface 23 and a flat locking surface 26. The stop anchor 50 has a complimentary recess 55 that on assembly snaps over the snap lock 21 signaling proper insertion depth. This embodiment also causes a bend in the anchored one or more sutures and can assist in holding the sutures in place under tension while insuring the stop anchor 50 cannot move relative to the sleeve 22 and bone tunnel.

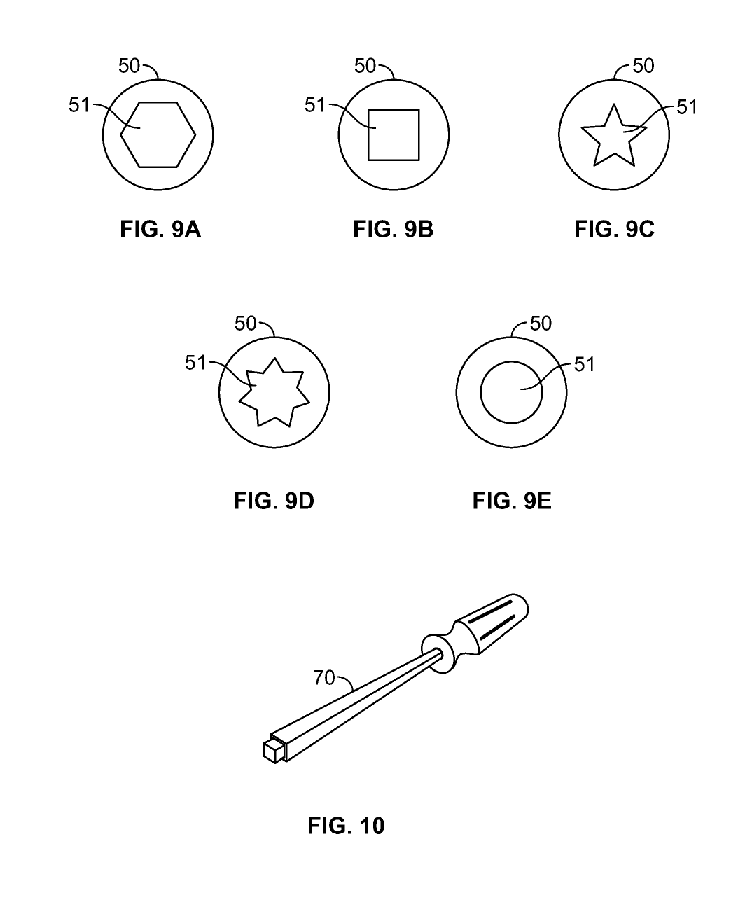

In FIGS. 9A-9E, the stop anchor 50 is shown in a top view with a recessed hole 51 in the end to receive an insertion tool 70. The insertion tool 70 can be a simple hand held device with a mating male end to fit into the stop anchor 50 as shown in FIG. 10 by way of example. The recessed hole 51 is a hexagonal, square, star, splined or round shape sized to receive the complimentary insertion tool 70. This device 70 allows the surgeon to safely and accurately place the stop anchor 50 in the sleeve 22 past or to the snap lock 21 and the snap lock 21 insures the stop anchor 50 stays in its fixed location as the tool 70 is withdrawn. This greatly simplifies the assembly while permitting the surgeon to simultaneously keep the sutures 40 tight. The procedure allows the surgeon to simply cut the exposed suture ends without tying any knots or making any additional incisions.

An important aspect of the present invention is the mechanism to hold the one or more sutures is a clamping action, pinching the sutures along a length of the suture between the mating surfaces of the stop anchor 50 and the sleeve 22. Accordingly, in the location of the suture, the mating surfaces have the sutures spacing the two parts, stop anchor 50 and sleeve 22 by the amount of the compressed sutures 40. This added thickness increases the interference of the parts locally along this compressed length and actually increases the gripping hold on the sutures 40. Where multiple sutures are used, it may require the diameter of the stop anchor 50 to be adjusted smaller to accommodate several of these locally higher interferences. Accordingly, the stop anchors 50 can be provided in kits. The kits of stop anchors can have different stop anchors 50 sized for 2, 4 or 6 sutures to insure fits are adapted for the number of sutures 40. This is not required in designs where the sleeve 22, stop anchor 50 or bone tunnel is adequately compliant. However, in applications of very hard bone tunnels or where the stop anchor 50 and the sleeve 22 have little resiliency such as when the sleeve and stop anchor are made of PEEK, polyether ether ketone, or other generally rigid plastic or metal, then different sized sleeves 22 or stop anchors 50 to accommodate different numbers of sutures 40 may be desirable. In any event, the clamping action of the stop anchor 50 and the opening 30 of the sleeve 22 has been preferably designed to provide uniform compression along the mating surfaces where the two parts are contacting and a locally higher clamping force can actually occur at the locations along the length of the suture between the two parts depending on the resilience or compliance of the stop anchor 50 and the sleeve 22. In either event, it is important the mating interference fit maintains a clamping action without creep or relaxation to reduce the clamping effect. The addition of the snap lock 21 feature provides a bend in the clamped suture 40 to add to the holding effect. As shown in FIGS. 1-6, the snap lock 21 when positioned at the end of the sleeve 22 provides a blocking action so the stop anchor 50 cannot shift above the sleeve 22 and the sleeve 22 is configured with internal tapers to help hold the stop anchor 50 in position. In FIG. 8, this snap lock 21 is positioned inwardly from either end of the sleeve 22; this insures the stop anchor 50 cannot move in either direction as it has an annular groove or recess locked onto the snap lock 21. This snap lock 21 feature is most preferable when the materials used are resilient or subject to creep or relaxation which could lower the clamping forces over time.

As shown in FIG. 1 to achieve the same two directional hold at the snap lock 21, the stop anchor 50 could have the recessed groove moved near an aft end of stop anchor 50 and in that case a small cap or end of the stop anchor 50 could be positioned slightly outwardly of the sleeve 22 at the end stop plate 32 on final assembly. In such a case, that portion exposed above the sleeve 22 should be kept to a minimum. This configuration is mentioned as an alternative way of using the principles and features of the embodiment of FIG. 8.

The present invention has been illustrated in relation to particular embodiments which are intended in all respects to be illustrative rather than restrictive. Those skilled in the art will recognize that the present invention is capable of many modifications and variations without departing from the scope of the invention. Accordingly, the scope of the present invention is described by the claims appended hereto and supported by the foregoing.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A suture anchor system for attachment of a soft tissue to a bone tunnel with one or more sutures, the suture anchor system comprising:
an elongated sleeve, the sleeve having a shaft with a central opening extending the length of the sleeve and having a snap lock extending radially inwardly from the central opening;
a stop anchor sized to be press fitted into the shaft central opening past the snap lock; and
wherein the one or more sutures secured to the soft tissue are held under tension as the stop anchor is press fit into the sleeve past the snap lock thereby fixing the one or more sutures along a length between the sleeve and the stop anchor.

2. The suture anchor system for attachment of a soft tissue to a bone tunnel with one or more sutures of claim 1 wherein the one or more sutures are further held at the stop anchor and the snap lock forming a bend in the suture.

3. The suture anchor system for attachment of a soft tissue to a bone tunnel with one or more sutures of claim 2 wherein the snap lock is located at an end of the sleeve and the stop anchor has an end positioned to be abutted against the snap lock.

4. The suture anchor system for attachment of a soft tissue to a bone tunnel with one or more sutures of claim 3 wherein one or more snap locks are located internal between opposing ends of the sleeve.

5. The suture anchor system for attachment of a soft tissue to a bone tunnel with one or more sutures of claim 4 wherein when the stop anchor is pressed into the sleeve at each snap lock the suture forms a bend.

6. The suture anchor system for attachment of a soft tissue to a bone tunnel with one or more sutures of claim 5 wherein the sleeve has a plurality of internal snap locks and the anchor has a plurality of complimentary locking ridges or recesses positioned to lock at a predetermined insertion depth of the anchor.

7. The suture anchor system for attachment of a soft tissue to a bone tunnel with one or more sutures of claim 1 wherein the elongated sleeve has a first end and a second end, the first end having a stop anchor plate extending radially outwardly from the sleeve, the stop anchor plate limits the insertion depth of the sleeve into the bone tunnel and lies on the exterior at the surface of the bone.

8. The suture anchor system for attachment of a soft tissue to a bone tunnel with one or more sutures of claim 1 wherein the shaft has a smooth exterior surface.

9. The suture anchor system for attachment of a soft tissue to a bone tunnel with one or more sutures of claim 1 wherein the shaft has a plurality of bone holding protrusions for fixing the sleeve in the bone tunnel.

10. The suture anchor system for attachment of a soft tissue to a bone tunnel with one or more sutures of claim 9 wherein the plurality of protrusions are spaced rings allowing the sleeve to be pressed into place.

11. The suture anchor system for attachment of a soft tissue to a bone tunnel with one or more sutures of claim 10 wherein the sleeve has a stop anchor plate to limit depth of insertion.

12. The suture anchor system for attachment of a soft tissue to a bone tunnel with one or more sutures of claim 1 wherein the stop anchor has a rounded, tapered or conical entry end or tip.

13. The suture anchor system for attachment of a soft tissue to a bone tunnel with one or more sutures of claim 1 wherein the stop anchor has a body extending from the entry end or tip to a distal end, the exterior surface between the ends sized to have an interference fit with internal surfaces of the shaft of the elongated sleeve over at least a length of 25 percent of the length of the anchor.

14. The suture anchor system for attachment of a soft tissue to a bone tunnel with one or more sutures of claim 13 wherein the stop anchor and the shaft have a contacting interference fit along the length of the body from a first contact located at or near the entry end or tip to the distal end.

15. The suture anchor system for attachment of a soft tissue to a bone tunnel with one or more sutures of claim 1 wherein on assembly the sutures are held and the stop anchor is pressed into the sleeve past the snap lock creating an audible or physical snap signaling a completed insertion.

16. The suture anchor system for attachment of a soft tissue to a bone tunnel with one or more sutures of claim 1 wherein the stop anchor has a recessed hole at a distal end, the recessed hole sized to accommodate an insertion tool.

17. The suture anchor system for attachment of a soft tissue to a bone tunnel with one or more sutures of claim 1 wherein the recessed hole is a hexagonal, square, star, splined or round shape sized to receive a complimentary insertion tool.

18. A method for anchoring soft tissue to a bone, the bone having a tunnel therethrough, said method comprising the steps of:
inserting an elongated sleeve in the tunnel to be maintained between said stop anchor and the tunnel;
attaching one or more sutures to the soft tissue;
receiving said sutures in the tunnel;
receiving a stop anchor in the tunnel by sliding disposing said stop anchor in said elongated sleeve in the tunnel, tensioning or tightening said sutures to remove slack and urging said sutures toward side walls of the tunnel with said stop anchor; and in response to said urging step, wedging said one or more sutures between said stop anchor and the elongated sleeve in the tunnel, thereby anchoring the soft tissue to the bone.

19. The method of claim 18 further comprising, in response to wedging said suture, the step of maintaining said suture between said stop anchor and said elongated sleeve.

20. The method of claim 18 wherein said wedging step comprises said stop anchor providing substantially uniform contact with said elongated sleeve.
